(19) United States
(12) Patent Application Publication
(10) Pub. No.: US 2024/0149456 A1
(43) Pub. Date: May 9, 2024

(54) DEVICE AND METHOD FOR PERFORMING MEASUREMENTS ON A WORKPIECE AS WELL AS MACHINING SYSTEM AND METHOD FOR MACHINING A WORKPIECE

(71) Applicant: Lessmuller Lasertechnik GmbH, Munchen (DE)

(72) Inventors: Eckhard LESSMÜLLER, Munchen (DE); Christian Truckenbrodt, Stockdorf (DE)

(21) Appl. No.: 18/479,216

(22) Filed: Oct. 2, 2023

(30) Foreign Application Priority Data
Nov. 4, 2022 (DE) 10 2022 129 218.5

(51) Int. Cl. B25J 9/16; B25J 15/00; G01B 9/02091; G01N 33/207

(52) U.S. Cl. CPC B25J 9/1679; B25J 9/1628; B25J 15/0019; G01B 9/02091; G01N 33/207

(57) ABSTRACT

The invention relates to a measuring device for performing measurements on a workpiece which are used to prepare and/or assess a weld seam produced by a welding device and having an initial portion and/or an end portion. The measuring device comprises a measuring unit comprising an optical coherence tomograph including a sample beam source, wherein the sample beam can be selectively focused on different measuring positions relative to a current machining position along the weld seam. The measuring device further comprises a fastening unit which is configured to attach at least the sample head to the welding device, and a control unit which is configured to dynamically adjust the leading beam and/or the trailing beam during machining along the weld seam. The invention further relates to a machining system, a method for performing measurements on a workpiece, and a method for machining a workpiece.

### CROSS-REFERENCE TO RELATED PATENT APPLICATION

This U.S. non-provisional patent application claims priority to German Patent Application No. 10 2022 129 218.5, filed on Nov. 4, 2022. The disclosure of the aforementioned priority application is incorporated herein by reference in its entirety.

### BACKGROUND

The invention relates to a device and a method for performing measurements on a workpiece which are used to prepare, monitor and/or assess welding performed by a robot-assisted movable machining head. The invention further relates to a machining system and a method for machining a workpiece.

Welding processes can be monitored using various methods. These include optical methods such as line triangulation, monitoring using a camera or optical coherence tomography. The latter offers a wide range of possible applications since it can be used to obtain precise height information of a workpiece to be machined at different locations. Other methods include distance monitoring, e. g. using inductive or optical sensors.

Monitoring measurements can be used, among other things, to implement seam tracking. In many cases, machining devices used for industrial purposes comprise a robot to which a machining head is attached that is movable relative to a workpiece to be machined by means of the robot. One challenge is to guide the machining head as precisely as possible. This means that the robot must be focused on a current welding position as precisely as possible.

As described, for example, in DE 10 2015 007 142 A1, a measuring device for optical coherence tomography can acquire measurement data along several sample lines, e. g. transverse to a machining direction and both in front of and behind a current machining position. On the one hand, this makes it possible to measure and characterize the workpiece to be machined where machining is to take place, and on the other hand, to check whether machining was successful and, for example, achieved the desired quality.

DE102016014564A1 also describes the use of multiple OCT sample lines. Here, a sample beam is coupled into a machining beam and focused on a workpiece. The sample beam is displaceable by a sample scanner in two spatial directions relative to the machining beam, which makes it possible to trace sample lines at different locations, in particular in front of a current machining position to measure the workpiece, at a current machining position to measure a melt pool, and behind a current machining position to characterize a weld seam formed.

Another aspect that can be significant in welding is the possible presence of a gap between workpieces to be joined. For this purpose, DE 10 2018 009 524 A1 describes an OCT-based measuring method in which targeted displacement of a sample beam relative to a current machining position and suitable monitoring measurements can be used to determine whether workpieces to be welded lie on top of each other as intended or whether there is an undesired gap between them.

Especially in gas-shielded welding, specifically in metal inert gas welding (MIG welding) and metal active gas welding (MAG welding), it became obvious that the use of measuring systems that are based on optical coherence tomography (OCT) can be superior to camera-based systems. The very bright welding process creates a significantly lower level of disturbance to OCT measurements than to camera-based triangulation systems. OCT also enables unidirectional seam tracking and/or seam monitoring.

For the purpose of seam tracking, a measurement is performed in front of the current machining position, making it possible to measure the workpiece or workpieces to be machined. For example, this helps to determine a joining edge or to monitor the correct positioning of a machining path. Furthermore, a measurement is performed behind the current machining position for seam monitoring. This makes it possible to measure and check a weld seam formed. Thus, a certain leading beam of the sample beam is usually used for seam tracking, and a certain trailing beam of the sample beam is used for seam monitoring.

Depending on the workpiece geometry, an area in which a weld seam is to be applied may be difficult to access by the welding device used and/or an OCT sample head used. In such situations, it may not yet be possible to make measurements in an initial portion of the weld seam with the desired leading beam or it may not yet be possible to make measurements in an end portion of the weld seam with the desired trailing beam, for example if the welding device and/or the sample head cannot be moved beyond the weld seam's starting point or end point due to limited accessibility. Accordingly, parts of the weld seam are produced without the seam being tracked and/or monitored fully.

Based on the prior art, the invention is based on the object of achieving improved seam tracking and/or seam monitoring.

This object is accomplished with a device for performing measurement on a workpiece having the features of claim 1, a machining system having the features of claim 8, a method for machining a workpiece having the features of claim 9, and a method for machining a workpiece having the features of claim 10.

In some embodiments, the invention relates to a measuring device for performing measurements on a workpiece which are used to prepare and/or assess a weld seam produced by a welding device and having an initial portion and/or an end portion. The measuring device comprises a measuring unit comprising an optical coherence tomograph including a sample beam source for producing a sample beam as well as a sample head by means of which the sample beam can be outcoupled, wherein the sample beam can be selectively focused on different measuring positions relative to a current machining position along the weld seam, so that, with respect to a machining direction, a leading beam and/or a trailing beam of a current measuring position are adjustable relative to the current machining position. The measuring device further comprises a fastening unit which is configured to attach at least the sample head to the welding device in such a way that the sample head is moved along with the welding device when the welding device is moving relative to the workpiece. In addition, the measuring device comprises a control unit which is configured to dynamically adjust the leading beam and/or the trailing beam during machining along the weld seam such that the leading beam increases along the initial portion and/or that the trailing beam decreases along the end portion.

In some embodiments, the invention further relates to a method for performing measurements on a workpiece, in particular by using the measuring device according to the invention, which are used to prepare and/or assess a weld seam produced on the workpiece and having an initial portion and/or an end portion. The method comprises the step of producing a sample beam using an optical coherence tomograph. The method further comprises the step of focusing the sample beam on different measuring positions during machining of the workpiece, wherein, with respect to a machining direction, a leading beam and/or a trailing beam of a current measuring position are adjusted relative to a current machining position along the weld seam. In addition, the method comprises the step of dynamically adjusting the leading beam and/or the trailing beam during machining along the weld seam such that the leading beam increases along the initial portion and/or that the trailing beam decreases along the end portion.

This enables using a dynamic leading beam and/or a dynamic trailing beam. This can improve seam tracking and/or seam monitoring. Specifically changing the leading beam and/or trailing beam in the initial portion and/or in the end portion allows the workpiece and the weld seam formed to be measured from the starting point of the weld seam and to its end point even where accessibility is limited. In particular, seam tracking can be performed from the first millimeter, and seam monitoring can be performed up to the last millimeter.

The workpiece may be a metallic component, e.g. a sheet metal component. The workpiece may also be composed of several individual workpieces to be welded together.

The measurements used to prepare the weld seam may be used for seam tracking. For example, it may be a measurement of a joining edge, a workpiece stack or a workpiece area in general where the weld seam is to be applied.

The measurements used to assess the weld seam may be used for seam monitoring. In particular, a height profile of the weld seam is generated at the relevant current measuring position. Such a height profile enables determination of whether or not the weld seam was formed without defects.

The measuring unit may comprise a sample scanner by means of which the sample beam is specifically displaceable in two spatial directions, in particular relative to a current machining position. The sample scanner may comprise at least two movable mirrors, each being displaceable in one spatial direction. The sample scanner may be attached in the sample head. The leading beam and/or the trailing beam may be adjustable by the sample scanner.

The sample beam source may comprise a broadband, low-coherence light source. The optical coherence tomograph may be spaced from the sample head. For example, the optical coherence tomograph may be independent of the welding device and stationary. In this case, the optical coherence tomograph may be connected to the sample head via an optical fiber. The optical coherence tomograph may have a sample arm and a reference arm, wherein the sample beam is optically guided in the sample arm and wherein a reference beam is optically guided in the reference arm, which may be caused to interfere with one another to perform optical coherence measurements.

The welding device may be a gas-shielded welding device, in particular a MIG/MAG welding device. The welding device may comprise a welding torch. The welding torch may comprise a wire feeder configured to deliver a welding wire to a current machining position.

The machining direction may run parallel to the weld seam, at least in sections. The machining direction may be variable, for example if the weld seam follows a winding, curved, angled or otherwise non-linear course.

The dynamic adjustment of the leading beam and/or trailing beam includes, without limitation, increasing and/or decreasing the leading beam and/or trailing beam as the welding device moves relative to the workpiece along the weld seam in the latter's initial portion and/or end portion. Specifically, the leading beam may be increased as the welding device moves along the initial portion in the machining direction. Alternatively or additionally, the trailing beam may be decreased as the welding device moves along the end portion in the machining direction. The measurement with the dynamic leading beam in the starting area may at least partially be performed before the welding device is activated. The measurement with the dynamic trailing beam in the end area may at least partially be performed after the welding device is deactivated.

### BRIEF SUMMARY

According to one embodiment, the control unit is configured to set the leading beam at a starting point of the weld seam substantially to zero and/or to set the trailing beam at an end point of the weld seam substantially to zero. This makes it possible to perform measurements right at the beginning of the weld seam and/or up to the end of the weld seam. Seam tracking may be performed along the entire seam even where accessibility is poor.

Useful measurement data can in particular be acquired even in the case of limited accessibility if the control unit is configured to gradually, in particularly linearly, increase the leading beam starting from a starting point of the weld seam along the initial portion and/or to gradually, in particularly linearly, decrease the trailing beam towards an end point of the weld seam along the end portion during machining along the weld seam (16). In particular, the leading beam and/or the trailing beam may be changed gradually in such a way that measurements are performed in the entire starting area and/or end area.

The measurement at the starting point of the weld seam may be performed before the welding device is activated. The measurement at the end point of the weld seam may be performed after the welding device is deactivated. In some embodiments, the welding device is activated after the initial portion is scanned. Alternatively or additionally, a scan of the end portion may be performed upon deactivation of the welding device. In other words, while the initial portion is scanned and the leading beam is dynamically increased, the welding device is at the starting point of the weld seam, and/or while the end portion is scanned and the trailing beam is dynamically decreased, the welding device is at the end point of the weld seam.

The control unit may be configured to set the leading beam and/or trailing beam to a substantially constant value during machining along the weld seam in a main machining portion of the weld seam, which differs from the initial portion and/or the end portion of the weld seam. This allows measurements to be performed in a simple and reliable manner during most of the machining operation. The dynamic adjustment of the leading beam may comprise a gradual increase to the substantially constant value, in particular starting from a leading beam of zero and/or starting from the starting point. The dynamic adjustment of the trailing beam may comprise a gradual decrease starting from the substantially constant value, in particular to a trailing beam of zero and/or to the end point.

Comprehensive information that can be used for precise seam tracking and/or seam monitoring may be obtained in particular if the control unit is configured to, during machining along the weld seam, deflect the sample beam at a current measuring position transversely and/or obliquely to the machining direction along a sample line and to control the optical coherence tomograph in such a way that a height profile can be generated along the sample line. Along the weld seam, measurements are performed in particular at different measuring positions along a sample line. In each of the leading beam and the trailing beam, a sample line may be used at the current measuring position.

Measurements in weld seams that are difficult to access both in front of and behind a current welding position will be possible in particular if the fastening unit is configured to attach the sample head to a first side of the welding device. The measuring device may further comprise at least one optical deflection element which, in an attached state of the fastening unit, is arranged on a second side of the welding device that is substantially opposite the first side, wherein the sample beam is guidable from the sample head on the first side to a measuring position located on the second side by means of the deflection element. The deflection element may comprise a mirror, prism or other suitable optical element. In some embodiments, the deflection element deflects the sample beam as a free beam. The deflection element may be stationary and/or immovable relative to the sample head and/or relative to the welding device. Alternatively, the deflection element may be movable, e.g. coordinated with the sample scanner.

The deflection element may comprise a curved mirror and/or an annular mirror. This allows a sample beam to be guided to different sides of the welding device in a simple manner. In some embodiments, it is not necessary to use a sample head that is movable relative to the welding device.

A high degree of precision of the measurements performed in different machining situations, including in weld seams that are difficult to access, may be achieved in particular if the fastening unit comprises a holder configured for stationary attachment to the welding device, and a support assembly supporting the sample head, the support assembly being movable relative to the holder such that a position of the sample head relative to the holder is variable.

The fastening unit may comprise at least one drive unit which is controllable by the control unit and configured to change the position of the support assembly relative to the holder, which, in an attached state of the fastening unit, makes the sample head movable to different sides of the welding device, in particular to a front and/or rear side with respect to the machining direction. This allows the sample head to be moved automatically to a suitable position to characterize a seam along its full length and/or to achieve seam tracking for the full length of the seam. For example, the sample head may be gradually and/or continuously and/or progressively moved from a first side to a second side as the welding device is moved along the weld seam. In this way, the sample head may be positioned in each case such that the sample beam can be focused on a current measuring position even where accessibility is limited.

In some embodiments, the invention further relates to a machining system and a method for welding a workpiece. The machining system comprises a welding device and a measuring device according to the invention, wherein at least the sample head of the measuring device is attached to the welding device by means of the fastening unit of the measuring device. The machining system may further comprise an industrial robot carrying the welding device and at least the sample head.

In some embodiments, the invention further relates to a method for machining a workpiece, in particular using a machining system according to the invention. The method comprises producing a weld seam having an initial portion and/or an end portion on a workpiece. The method further comprises the steps of producing a sample beam, focusing the sample beam on different measuring positions, and dynamically adjusting the leading beam and/or trailing beam according to the method for performing measurements on a workpiece as described herein.

In some embodiments, the invention relates to a device for performing measurements on a workpiece which are used to prepare, monitor and/or assess welding performed by a robot-assisted movable machining head. The device may comprise a robot control system configured to generate control signals for robot-assisted movement of the machining head. The device may further comprise a measuring unit. The measuring unit comprises a measurement sensor system configured to perform measurements on the workpiece and acquire measurement data.

The device may further include an evaluation unit configured to determine workpiece-specific position information from acquired measurement data, on the basis of which real-time position control for the machining head can be performed. In addition, the device may comprise an interface configured to transmit the workpiece-specific position information determined by the evaluation unit to the robot control system. The robot control system may be configured to perform position control for the machining head based on the position information.

The inventors have become aware that robots carrying machining heads do have communication interfaces that make real-time position manipulation possible but that such interfaces are often too slow to implement vibration-free control coming from the measuring system. The following communication interfaces may be used for example: “Guided Motion” or “Robot Sensor Interface” (“RSI”). As position control is performed by the robot control system itself, faster and more precise control is possible. This may improve seam tracking. The measuring unit transmits current position information to the robot control system, allowing the robot control system to adjust the position quickly and precisely on the basis of this information. This enables calculations of the extent to which the robot is to be moved to change the position of the machining head in accordance with the position information to be made directly in the robot control system. This helps to achieve at least substantially vibration-free control.

The measuring unit may be the measuring unit of a measuring device as described above. This means that the above statements concern optional embodiments of the device or measuring unit described herein. In some embodiments, the measurement sensor system may alternatively or additionally use measurement principles that differ from optical coherence tomography. For example, the measurement sensor system may be based on line triangulation and/or include a camera and/or include a distance sensor, e.g. an inductive and/or an optical distance sensor. Components of the measurement sensor system may be movable by an actuator. In some embodiments, the measuring unit may comprise an optical coherence tomograph including a sample beam source for producing a sample beam as well as a sample head by means of which the sample beam can be outcoupled, wherein the sample beam can be selectively focused on different measuring positions relative to a current machining position.

Position control may comprise PID control. The robot control system may be part of a robot such as an industrial robot. The robot control system may be operable independently of the measuring unit. A connection between the measuring unit and the robot control system, through which the position information is transmittable, may be established via a communication interface of the robot. In other words, the interface of the measuring unit may be connected to a communication interface of the robot.

Position control is in particular performed with a response time that exceeds a response time of a communication interface of the robot. In particular, position control can be faster than it would be if position control were performed by the measuring unit. In this case, a transmission time via the communication interface and a computing time in the measuring unit would add up. In particular, real-time control according to the invention may include a response time of up to 10 ms, of up to 5 ms or even of up to 1 ms.

The evaluation unit may be separate from the robot control system. The evaluation unit is, in particular, not a component of the robot control system and/or the robot. The evaluation unit may be configured in a processing unit of the measuring unit, e.g. in a processor, controller, field programmable gate array, control system or the like, combined with a suitable volatile and/or non-volatile storage medium, where applicable.

The machining head may comprise a welding device and/or be configured as a welding device. The machining head and/or the welding device may be a gas-shielded welding device, in particular a MIG/MAG welding device. The machining head and/or the welding device may comprise a welding torch. The welding torch may comprise a wire feeder configured to deliver a welding wire to a current machining position.

Seam tracking may be performed very precisely, in particular, if the position information includes a current position of an edge. The position of the edge may be determined using a suitable measuring method. This position may then be passed on to the robot control system. According to the invention, a robot position on the edge is then controlled by the robot control system, making it possible to implement very quick and low-vibration control.

In some embodiments, the measuring unit is configured to perform at least one measurement at a predeterminable measuring position, with the robot control system being configured to define the measuring position. This allows a measuring position to be set quickly and reliably since the robot control system can determine the measuring position based on the set machining position without any latencies of a communication interface having an effect. For example, the robot control system may take the position information into account to define the measuring position. This means that a measurement can be performed relative to an actual current machining position.

A measurement can be adapted to a current machining situation quickly and precisely in particular if the robot control system is configured to adjust a leading beam and/or trailing beam of the measuring position relative to a current machining position. With regard to the options of adjusting a leading beam and/or a trailing beam, reference is made to the above explanations. However, the leading beam and/or the trailing beam may be adjustable accordingly for any of the mentioned measuring methods, the use of an OCT sample beam is only one possible option. It is understood that, according to this embodiment, the leading beam and/or the trailing beam can be adjusted directly by the robot control system, meaning that the fact that the robot control system has the information required for adjusting the leading beam and/or the trailing beam on the basis of the position information and the position control based on this information can be used to advantage.

The robot control system may further be configured to dynamically adjust the leading beam and/or the trailing beam during machining along a weld seam having an initial portion and an end portion such that the leading beam increases along the initial portion and/or that the trailing beam decreases along the end portion. This allows a weld seam to be measured very precisely since its initial portion and/or end portion are scanned. With regard to the options of dynamically adjusting a leading beam and/or a trailing beam, reference is made to the above explanations. However, the leading beam and/or the trailing beam may be dynamically adjustable accordingly for any of the mentioned measuring methods, the use of an OCT sample beam is only one possible option.

A high degree of variability with regard to obtainable measurement data may be achieved in particular if the measuring unit is configured to perform measurements at several measuring positions lying on a geometric measurement figure, in particular at least one measurement line. In such case, the robot control system may be configured to predetermine a position and/or orientation and/or scanning density of the geometric measurement figure. Since information on the previous and/or future pathway of the weld seam may be available in the robot control system, measurement parameters can be adjusted, taking the relevant application into account. The robot control system may in particular perform the parameterization of the measurement figure, e.g. rotation, position, elongation, compression, deformation, etc.

Position control performed in a robot control system may also be used in a machining system. According to one aspect, a machining system hence comprises a device as described above, a robot controllable by the robot control system, and a machining head attached to the robot and movable by means of the robot. In particular, the robot control system is part of the robot. The robot including its robot control system may be a robot operable independently of the measuring unit, in particular an industrial robot.

In one aspect, a method may further be provided for performing measurements on a workpiece which are used to prepare, monitor and/or assess welding performed by a robot-assisted movable machining head. This may be performed in particular by the device described above. The method comprises a step of generating control signals for the robot-assisted movement of the machining head by means of a robot control system, a step of performing measurements on the workpiece and acquiring measurement data, a step of determining workpiece-specific position information from the acquired measurement data, on the basis of which real-time position control for the machining head can be performed, a step of transmitting the determined workpiece-specific position information to the robot control system, and a step of performing position control for the machining head based on the position information by means of the robot control unit.

In one aspect, a method may further be provided for machining a workpiece, in particular by means of the aforementioned machining system. The method comprises a step of generating control signals for the robot-assisted movement of a machining head by means of a robot control system, a step of performing a welding operation on the workpiece by means of the machining head in accordance with the generated control signals, a step of performing measurements on the workpiece and acquiring measurement data, a step of determining workpiece-specific position information from the acquired measurement data, on the basis of which real-time position control for the machining head can be performed, a step of transmitting the determined workpiece-specific position information to the robot control system, and a step of performing position control for the machining head based on the position information by means of the robot control unit.

As mentioned, these aspects are based on the awareness that robots carrying machining heads do have communication interfaces that make real-time position manipulation possible but that such interfaces are often too slow to implement vibration-free control coming from the measuring system. The aforementioned methods may help to achieve at least substantially vibration-free control.

In particular, it is pointed out that all features and properties described with respect to devices as well as procedures can be applied mutatis mutandis to methods according to the invention and are applicable in the sense of the invention and deemed to be disclosed as well. The same applies vice versa. This means that structural features, i.e. features according to the device, mentioned with respect to methods can also be taken into account, claimed as well as deemed to be disclosed within the scope of the device claims.

Below, the present invention is described by way of example with reference to the accompanying figures. The drawing, the specification and the claims contain combinations of numerous features. The skilled person will appropriately consider the features also individually and use them in useful combinations within the scope of the claims.

### BRIEF DESCRIPTION OF THE DRAWINGS

FIG. 1 is a schematic representation of a machining system comprising a measuring device;

FIG. 2 is a schematic representation of a leading beam of measuring positions considered by means of the measuring device during machining along a weld seam;

FIG. 3 is a schematic representation of a trailing beam of measuring positions considered by means of the measuring device during machining along the weld seam;

FIG. 4 is a schematic representation of an alternative measuring device;

FIG. 5 is a schematic flow chart of a method for machining a workpiece;

FIG. 6 is a schematic representation of another machining system;

FIG. 7 is a schematic flow chart of a method for performing measurements on a workpiece; and

FIG. 8 is a schematic flow chart of a method for machining a workpiece.

### DETAILED DESCRIPTION

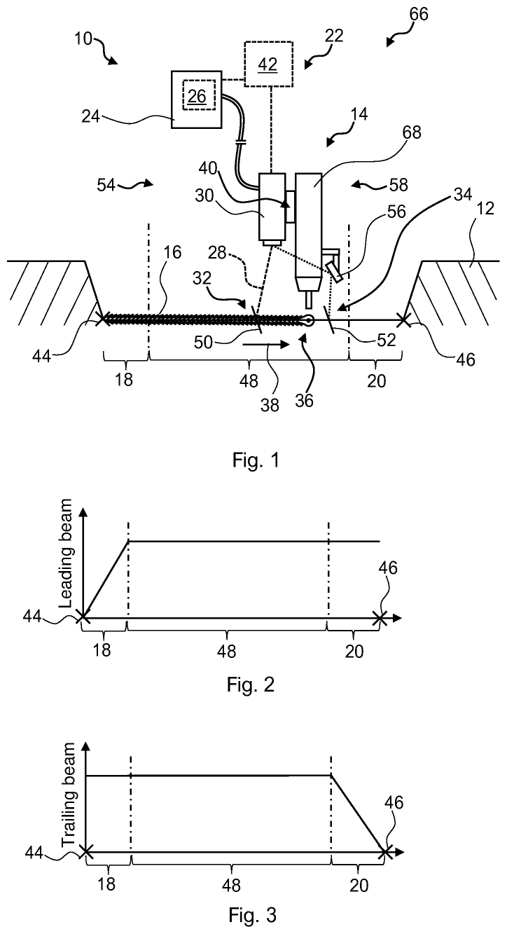

FIG. 1 is a schematic representation of a machining system 66 comprising a measuring device 10. The machining system 66 comprises a welding device 14 comprising a welding torch 68. The welding torch 68 is configured as a MIG/MAG welding torch, for example. The welding device 14 is configured to weld a workpiece 12, wherein a weld seam 16 is formed by the welding device 14 machining the workpiece 12 in a machining direction 38. The workpiece 12 may be configured in any manner and may comprise, for example, two separate components to be connected to each other along the weld seam 16. The welding device 14 is attached to an industrial robot (not illustrated), for example, by means of which the welding device is movable relative to the workpiece 12 along a machining path. Alternatively or additionally, the welding device 14 may be stationary, and the workpiece 12 may be movable to produce the relative movement.

In the illustrated case, accessibility to the weld seam 16 is limited because of the geometry of the workpiece 12. In FIG. 1, this is shown only schematically. Limited accessibility may consist, for example, in the fact that the weld seam 16 extends from a starting point 44 to an end point 46, each of which is very close to or directly adjacent to workpiece portions that protrude from the weld seam such that the welding device 14 cannot be moved arbitrarily beyond the starting point 44 and/or the end point 46 due to a risk of collision.

In addition to the welding device 14, the machining system 66 further comprises a measuring device 10. The measuring device 10 is an OCT measuring device. The measuring device 10 comprises a measuring unit 22 including a sample head 30 that is attached to the welding device 14, in particular to the welding torch 68, with a fastening unit 40 of the measuring device 10. The measuring unit 22 further comprises an optical coherence tomograph 24 including a sample beam source 26, which are shown purely schematically and have a generally known structure. Reference is made in this regard, for example, to DE 10 2016 014 564 A1.

The measuring device 10 is configured to focus a sample beam 28 produced by the sample beam source 26 specifically on different measuring positions 32, 34. At the measuring positions 32, 34, the sample beam 28 is in each case displaceable transversely or obliquely to the machining direction 38 and/or transversely or obliquely to the weld seam 16 along a respective measuring line 50, 52. In the case illustrated by way of example, at least two different measuring positions 32, 34 relative to a current machining position 36 are used.

A first measuring position 32 is located behind the current machining position 36 in the machining direction 38, which means that the first measuring position 32 has a trailing beam. This allows the weld seam 16 to be measured after being formed, which enables, for example, quality assurance and/or process control and/or process regulation in dependence of a property of the weld seam 16. The measurement with the trailing beam enables seam monitoring.

A second measuring position 34 is located in front of the current machining position in the machining direction 38, which means that the second measuring position 34 has a leading beam. This allows the workpiece 12 to be measured before being machined, which makes it possible, for example, to determine whether a joining edge of workpieces to be joined is positioned correctly, whether components lie on top of each other without gaps and/or whether an intended machining path, which the welding device 14 travels along, is positioned correctly.

During welding, the sample beam 28 is continuously displaced at different measuring positions 32, 34 along suitable measuring lines 50, 52. For example, the sample beam 28 is moved alternately in front of and behind the current machining position 36. Thus, measurements with the leading beam and measurements with the trailing beam take place repeatedly during machining. However, it is understood that in other embodiments measurements may be performed only with the leading beam or only with the trailing beam. In principle, there may be several sample heads, for example one sample head for the leading beam and one for the trailing beam, and/or several sample beams.

In order to enable seam tracking and/or seam monitoring that are/is as complete as possible, the measuring device 10 is operated during machining as described below. Starting from the starting point 44, machining is initially performed in an initial portion 18, then in a main machining area 48, and subsequently in an end portion 20 up to the end point 46. The leading and trailing beams of the current measuring position 32, 34 are adjusted dynamically. This is illustrated in FIG. 2 for the leading beam and in FIG. 3 for the trailing beam.

The leading beam is first set to zero at the starting point 44. This means that the workpiece 12 can also be measured directly at the starting point. The leading beam is then increased gradually, for example linearly. In the example shown, the welding device 14 will not be activated before that, i.e. the initial portion 18 is scanned first, before machining begins. During this process, the welding device 14 may be stationary or move at a lower speed than what corresponds to the increase in the leading beam. This allows seam tracking also for the first millimeters or centimeters of the weld seam 16 because the workpiece 12 can be measured completely for the entire weld seam 16 in front of the current machining position 36.

Similarly, the trailing beam is decreased gradually, for example linearly, as the end point 46 is approached in the end portion 20. This may occur upon deactivation of the welding device 14, i.e. upon completion of the formation of the weld seam 16. During this process, the welding device 14 may be stationary or move at a lower speed than what corresponds to the decrease in the trailing beam. This allows seam monitoring also for the final millimeters or centimeters of the weld seam 16 because the weld seam 16 can be scanned completely behind the current machining position 36.

In the main machining portion 48, for example, measurements are performed with a constant leading beam and/or a constant trailing beam as shown. The leading beam and the trailing beam may be the same or different. The leading beam is increased to its target value for the main machining portion 48 along the initial portion 18, the trailing beam is decreased along the end portion 20 based on its target value for the main machining portion 48.

The measuring device 10 comprises a control unit 42 configured to make the described adjustment of the leading beam and/or trailing beam. The control unit 42 comprises, for example, a processor, a random-access memory, a non-volatile memory, and corresponding programming. The control unit 42 may be configured, for example, to control a sample scanner (not illustrated) of the measuring device 10, in particular in the sample head 30, to displace the sample beam 28 as described.

As an optional feature, the measuring device 10 includes a deflection element 56 by means of which the sample beam 28 can be guided from a first side 54 of the welding device 14 to a second side 58 of the welding device. The deflection element 56 comprises or is configured as a curved mirror and/or an annular mirror, for example. An annular and, in particular, suitably curved mirror may be shaped such that a displacement of the sample beam 28 by means of a sample scanner of the measuring device 10 is translated into a displacement on the workpiece 12, at least substantially independently of whether the sample beam 28 is focused directly on the workpiece 12 or passes through the deflection element 56.

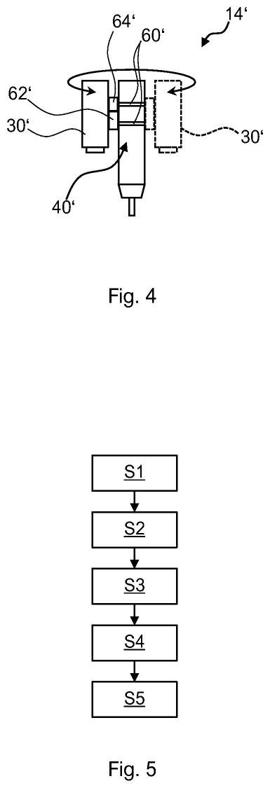

FIG. 4 is an alternative embodiment of a measuring device 10′. For differentiation purposes, the reference signs in FIG. 4 are provided with an apostrophe. The measuring device 10′ is configured as described above but differs with regard to its fastening unit 40′. The fastening unit 40′ comprises a holder 60′ that is fixedly attached to a welding device 14′. The fastening unit 40′ further comprises a support assembly 62′ that supports a sample head 30′ of the measuring device 10′ and is movable relative to the holder 60′. A drive unit 64′ is provided for this purpose, which is controllable by a control unit of the measuring device 10′. The drive unit 64′ allows the sample head 30′ to be moved to different positions by moving the support assembly 62′ relative to the holder 60′. The sample head 30′ is thus movable to different, in particular opposite, sides of the welding device 14′. This also makes it possible to specifically focus the sample beam 28 on a workpiece on any side of the welding device 14′, even where accessibility is limited.

FIG. 5 is a schematic flow chart of a method for machining a workpiece 12. The operation of the method is also apparent from the above exemplary representation. Generally speaking, the method comprises a step S1 of producing a weld seam 16 having an initial portion 18 and/or an end portion 20 on the workpiece 12.

The method further comprises a step S2 of producing a sample beam 28 using an optical coherence tomograph 24.

In addition, the method comprises a step S3 of focusing the sample beam 28 on different measuring positions 32, 34 during machining of the workpiece 12, wherein, with respect to a machining direction 38, a leading beam and/or a trailing beam of a current measuring position 32 are adjusted relative to a current machining position 36 along the weld seam 16.

The method further comprises a step S4 of dynamically adjusting the leading beam and/or the trailing beam during machining along the weld seam 16 such that the leading beam increases along the initial portion 18 and/or that the trailing beam decreases along the end portion 20.

The steps S2 to S4 may be separately part of a method for performing measurements on a workpiece 12 which are used to prepare and/or assess a weld seam 16 produced on the workpiece 12 and having an initial portion 18 and/or an end portion 20.

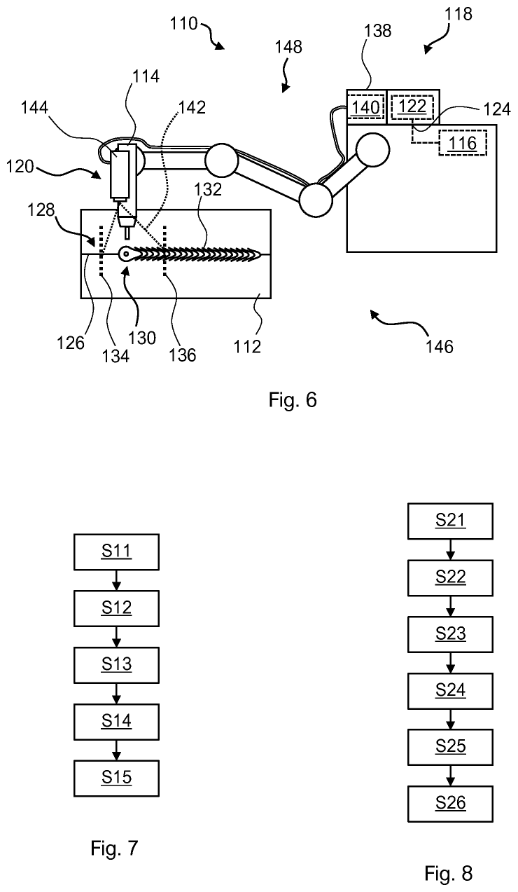

FIG. 6 shows another machining system 146. The further machining system 146 may be basically the same as the machining system 66 described above, and vice versa. In the following, the machining system 146 will be described with reference to features relating to position control. This aspect may also be provided in the machining system 66 described above, just as aspects of the machining system 66 described above may be present in the machining system 146. The description with reference to two machining systems 66, 146 serves the twofold purpose of illustrating that the aspects may be used independently of each other and of providing an understanding of the aspects, wherein it is understood, however, that in some embodiments they are combined.

The machining system 146 comprises a robot 148 supporting a machining head 114 that is movable by means of the robot 148. The machining system 146 further comprises a device 110 comprising a robot control system 116 of the robot 148 as well as a measuring unit 118. The robot control system 116 is configured to generate control signals for the robot 148 to move the machining head 114. In the illustrated case, the machining head 114 is a welding torch by analogy to the case described above. With the machining head 114, a workpiece 112 is machinable. The present case relates to welding along an edge 126, which serves the purpose of, for example, connecting two components to one another. In this case, the workpiece 112 may comprise several individual parts/components.

The measuring unit 118 comprises a measurement sensor system 120 configured to perform measurements on the workpiece 112 and acquire measurement data. The measurement sensor system 120 may include any suitable sensors and/or sample light sources for acquiring 2D data and/or 3D data. Examples include line triangulation sensors, one or more cameras, particularly including planar illumination, one or more distance sensors, or the like. Suitable distance sensors may be optical and/or inductive sensors. In the present case, the measurement sensor system 120 is configured to perform measurements at different measuring positions 128 that differ from a current machining position 130 and/or a tool center point (TCP). The measuring position 128 may be adjustable by means of the measurement sensor system 120 itself and/or using additional components such as actuators, motors, scanners, etc. For example, if a sensor providing few or no parameterization options is used, it may be attached to be dynamically movable to allow different measuring positions 128 to be set.

In the exemplary embodiment described herein, the measurement sensor system 120 is an OCT measurement sensor system. The measuring unit 118 comprises an optical coherence tomograph 138 having a sample beam source 140 for producing a sample beam. The measuring unit 118 further comprises a sample head 144 by means of which the sample beam 142 can be outcoupled and selectively focused on different measuring positions relative to a current machining position. In this respect, reference is also made to the above explanations regarding the operation of the measuring device 10. In particular, a leading beam and/or a trailing beam of the measuring position 128 are adjustable also for the measuring unit 118.

The measuring unit 118 is specifically configured to perform measurements at several measuring positions 128 lying on a geometric measurement FIG. 134, 136. As an example, a first geometric measurement FIG. 134 is a first measurement line that scans the edge 126 or the workpiece 112 in general in front of a current machining position 130. Further, a second geometric measurement FIG. 136 is a second measurement line that scans a weld seam 132 formed when machining the workpiece 112. For example, scanning may be performed transversely to a machining direction and/or transversely to the weld seam 132.

Measurement lines represent only one example of possible measurement FIGS. 134, 136. Alternatively or additionally, measurement FIGS. 134, 136 may also be defined by measuring positions 128 lying on any geometric figures such as circles, ellipses, polygons, etc.

The measuring unit 118 further comprises an evaluation unit 122. The evaluation unit 122 is configured to determine workpiece-specific position information from acquired measurement data, on the basis of which real-time position control for the machining head 114 can be performed. The position information may, for example, be obtained from the measuring unit 118 on the basis of edge detection. The position information is, for example, a current position of the edge 126. The position information can thus be used to determine where machining is to be performed. The position information can in particular be used to perform seam tracking.

The measuring unit 118 further comprises an interface 124 configured to transmit the workpiece-specific position information determined by the evaluation unit 122 to the robot control system 116. The robot 148 may include a communication interface that is connected to the interface 124.

To perform position control in accordance with the position information, in the present case, control is carried out in the robot control system 116. This allows the position of the machining head 114 to be directly adopted by the robot control system 116 itself. Control via the communication interface of the robot 148 could cause vibration in position control, even if the communication interface operates in real time. Known robot communication interfaces, even if operating in real time, are usually too slow to control the position of the machining head 114 on the basis of the measuring unit 118 without causing vibration.

The robot control system 115 may also perform other functions. In the present case, the robot control system 116 is configured to adjust the leading beam and/or the trailing beam of the measuring position relative to the current machining position 130. Further, the robot control system 116 specifies a position and/or orientation and/or scanning density of the geometric measurement FIG. 134, 136. The transfer of the position information may enable the robot control system 116 to perform the positioning of the machining head 114 as well as the positioning of the sample beam 142 or the measuring position 128 in general. Thus, seam tracking is calculated in the robot control system 116, and process monitoring is significantly controlled by the robot control system 116. In particular, this allows for very precise and quick adjustment of those parameter values that depend on the position of the machining head 114 actually set.

FIG. 7 illustrates a method for performing measurements on a workpiece 112 which are used to prepare, monitor and/or assess welding performed by a robot-assisted movable machining head 114. The method is performed, for example, with the device 110. The operation of the method is also apparent from the above exemplary representation.

A step S11 comprises generating control signals for robot-assisted movement of the machining head 114 by means of a robot control system 116. A step S12 comprises performing measurements on the workpiece 112 and acquiring measurement data. A step S13 comprises determining workpiece-specific position information from the acquired measurement data, on the basis of which real-time position control for the machining head 114 can be performed. A step S14 comprises transmitting the determined workpiece-specific position information to the robot control system 116. A step S15 comprises performing position control for the machining head 114 based on the position information by means of the robot control system 116.

FIG. 8 illustrates a method for machining a workpiece 112. The workpiece 112 is machined in particular with the machining system 146. The operation of the method is also apparent from the above exemplary representation. A step 21 comprises generating control signals for robot-assisted movement of a machining head 114 by means of a robot control system (116). A step 22 comprises performing a welding operation on the workpiece 112 by means of the machining head 114 in accordance with the generated control signals. A step 23 comprises performing measurements on the workpiece 112 and acquiring measurement data. A step 24 comprises determining workpiece-specific position information from the acquired measurement data, on the basis of which real-time position control for the machining head 114 can be performed. A step 25 comprises transmitting the determined workpiece-specific position information to the robot control system 116. A step 26 comprises performing position control for the machining head 114 based on the position information by means of the robot control system 116.

1. A device for performing measurements on a workpiece which are used to prepare, monitor and/or assess welding performed by a robot-assisted movable machining head, comprising:
a robot control system configured to generate control signals for robot-assisted movement of the machining head;
a measuring unit comprising:
a measurement sensor system configured to perform measurements on the workpiece and acquire measurement data;
an evaluation unit configured to determine workpiece-specific position information from acquired measurement data, on the basis of which real-time position control for the machining head can be performed; and
an interface configured to transmit the workpiece-specific position information determined by the evaluation unit to the robot control system;
wherein the robot control system is configured to perform position control for the machining head based on the position information.

2. The device of claim 1, wherein the position information includes a current position of an edge.

3. The device of claim 1, wherein the measuring unit is configured to perform at least one measurement at a predeterminable measuring position, and wherein the robot control system is configured to define the measuring position.

4. The device of claim 3, wherein the robot control system is configured to adjust a leading beam and/or trailing beam of the measuring position relative to a current machining position.

5. The device of claim 4, wherein the robot control system is configured to dynamically adjust the leading beam and/or the trailing beam during machining along a weld seam having an initial portion and an end portion such that the leading beam increases along the initial portion and/or that the trailing beam decreases along the end portion.

6. The device of claim 3, wherein the measuring unit is configured to perform measurements at several measuring positions lying on a geometric measurement figure, in particular at least one measurement line; and wherein the robot control system is configured to specify a position and/or orientation and/or scanning density of the geometric measurement figure.

7. The device of claim 1, wherein the measuring unit may comprise an optical coherence tomograph including a sample beam source for producing a sample beam as well as a sample head by means of which the sample beam can be outcoupled, wherein the sample beam can be selectively focused on different measuring positions relative to a current machining position.

8. A machining system, comprising:
a device of claim 1;
a robot controllable by the robot control system; and
a machining head that is attached to the robot and movable by means of the robot.

9. A method for performing measurements on a workpiece which are used to prepare, monitor and/or assess welding performed by a robot-assisted movable machining head, in particularly performed by a device of claim 1, comprising:
generating control signals for robot-assisted movement of the machining head by means of a robot control system;
performing measurements on the workpiece and acquiring measurement data;
determining workpiece-specific position information from the acquired measurement data, on the basis of which real-time position control for the machining head can be performed;
transmitting the determined workpiece-specific position information to the robot control system; and
performing position control for the machining head based on the position information by means of the robot control system.

10. A method for machining a workpiece, in particular with a machining system of claim 8, comprising:
generating control signals for robot-assisted movement of a machining head by means of a robot control system;
performing a welding operation on the workpiece by means of the machining head in accordance with the generated control signals;
performing measurements on the workpiece and acquiring measurement data;
determining workpiece-specific position information from the acquired measurement data, on the basis of which real-time position control for the machining head can be performed;
transmitting the determined workpiece-specific position information to the robot control system; and
performing position control for the machining head based on the position information by means of the robot control system.
